(19) United States
(12) Patent Application Publication
(10) Pub. No.: US 2015/0025481 A1
(43) Pub. Date: Jan. 22, 2015

(54) Pocketable biodegradable powder application device

(71) Applicant: Darlene Tyminski Ricketts, West Rutland, VT (US)

(72) Inventor: Darlene Tyminski Ricketts, West Rutland, VT (US)

(21) Appl. No.: 13/987,360

(22) Filed: Jul. 17, 2013

(51) Int. Cl. A61M 35/00

(52) U.S. Cl. CPC A61M 35/003; A61M 2202/064; A61M 2210/04; A61B 2017/00004
USPC 604/309

(57) ABSTRACT

A pocketable biodegradable powder application device having a cardboard backer component affixed to the backside of which is a cardboard handle component and having adjacent to the frontside of the backer component, a first piece component made up of cotton batten material and having anteriorly adjacent to the first piece component, a frontal membrane component through which grains of cornstarch powder material held between it and the first piece component can pass whenever the device is pressed against the skin of a hospital patient, with the backer component, the first piece component and the frontal membrane component all being adhesively affixed to one another about the perimeters of each, once powder material would have been positioned in between the first piece component and the frontal membrane component; and with the intact application device being readily amenable to being held within a pocket unit made of paper material.

### PRIOR OR PARENT APPLICATIONS

There are no prior or parent applications as respects the invention.

### FEDERALLY SPONSORED RESEARCH AND DEVELOPMENT

The invention is not the subject of any federally sponsored research and development.

### BACKGROUND OF THE INVENTION

1. Field of the Invention

The invention relates to that coterie of devices utilized for the purpose of applying powdered substances to portions of the human body.

2. Related Art

The herewith submitted Information Disclosure Statement references art that is related to the invention. Such art however does not, respectfully submitted, anticipate the invention.

### A SUMMARY OF THE INVENTION

### 1. A Brief Description of the Invention

The invention is a biodegradable powder application device, having a sturdy cardboard backer component to the backside of which there is adhesively affixed, e.g. with glue material, a cardboard handle component. The handle component is foldable about a long axis thereof such that when roughly half of the handle component is adhesively affixed to the backer component, the remaining unaffixed half or so portion thereof is amenable, when folded outwardly, to being gripped with a person's thumb and forefinger for application purposes. Adjacent the frontside of the backer component is a first piece component made of garden variety cotton batten material. Positioned anterior to the first piece component is a frontal membrane component made of material being e.g. a portion of the outer membrane of a Johnson and Johnson SURGIPAD® unit. Sandwiched between the first piece component and the frontal membrane component is a batch of dry cornstarch powder or garden variety baby powder; typically, a teaspoon full of such powder. The frontal membrane component is sufficiently porous to enable the passage through it; but only under pressure resulting from gently tapping a portion of the outer skin surface area of a person's body with the intact invention; of the powder onto the so-tapped surface area portion. The outer perimeters of the frontal membrane component, the first piece component and the cardboard backer component are all adhesively affixed each to one another with e.g. a glue material and/or an adhesive tape sealant material. Also, the powder application device is optionally amenable to being held within a pocket unit being wholly made up of biodegradable paper material.

### 2. Objects of the Invention

Oftentimes, it becomes necessary to apply powders to certain outer skin areas of the body of a human hospital patient bedridden and stricken with a rash or rashes and/or so-called bedsores (decubitus ulcers) in various stages of development in order to heal or, at least, somewhat ameliorate the adverse effects of such maladies.

Difficulty in achieving the same is often however encountered because such powder when so applied, either by way of sprinkling it on such so adversely affected areas of the body or via currently in vogue application devices, has a tendency to either clump or become inefficiently dispersed to nearby unaffected areas of the body. Such application undertakings invariably result in greatly diminished efficacy insofar as any desired therapeutic effect to be sought with resort to any such application is concerned. Resort however to utilization of the presently invented device for such purposes, serves to veritably, wholly obviate any propensity, upon application, for any such clumping or undesirable dispersion. One needs only to gently tap the so affected area of a patient's body with the invention and powder is instantly released through the frontal membrane component thereof directly onto the so-affected area with no clumping and no dispersion.

Also, with an eye towards concerns for safety and sanitation with respect to such application undertakings, the invention readily amenable to being equipped with sandwiched-in powder as noted above, can simply be discarded after just one use. The invention is very inexpensive to manufacture and unlike any other sort of similar and arguably inexpensive powder application devices, it is wholly biodegradable. It is this unique feature of biodegradability that serves to notably address such safety and sanitation concerns especially as respects the matter of discarding the device post-application. The device is readily amenable to being held within a pocket unit made of paper material. It is removed therefrom when needed for use and simply reinserted therein after use in preparation for safe and sanitary discarding of the same.

In closing, respectfully submitted, the invention in respect of facilitating the safe, inexpensive and highly efficient application of powders to such so adversely affected surface areas of a hospital patient's person, is clearly new, useful and unique.

### A DESCRIPTION OF THE DRAWINGS

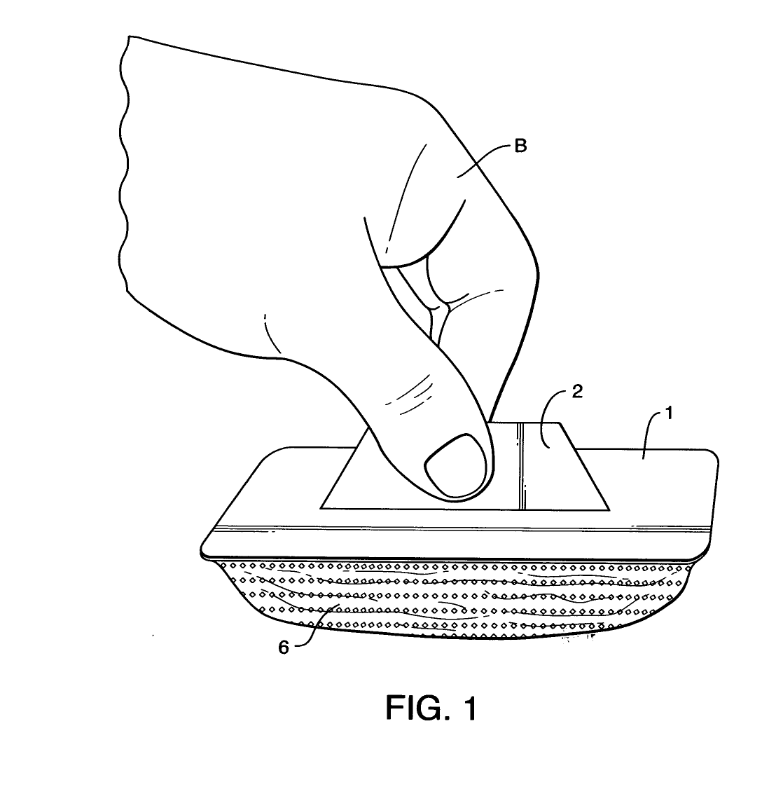

1. FIG. 1 is a perspective view of the powder application device being held by the thumb and forefinger of a person's hand.

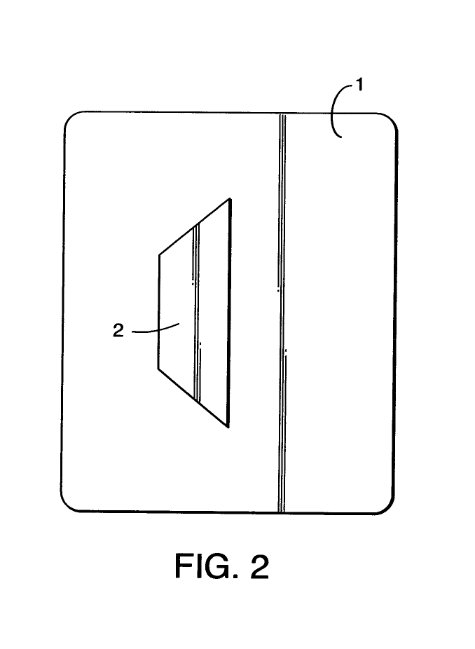

2. FIG. 2 is a rear plan view of the backer component of the application device to which there is affixed a handle component.

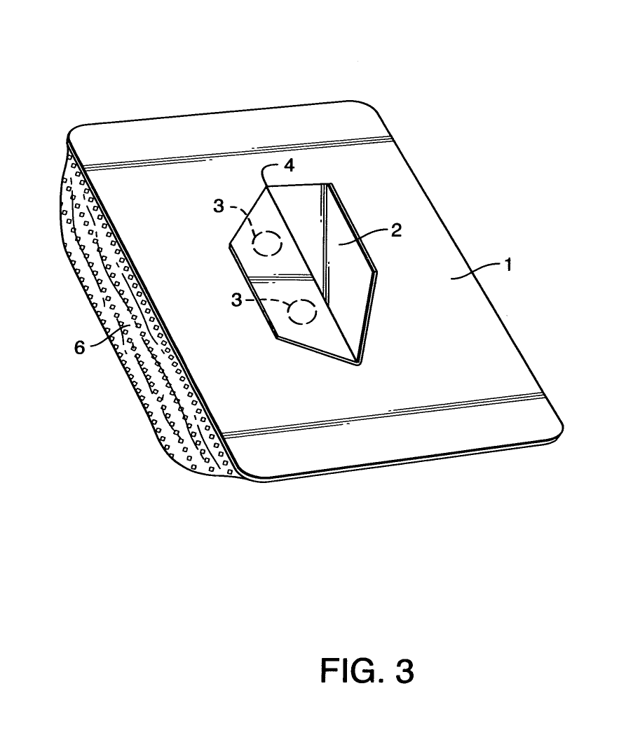

3. FIG. 3 is an isolated perspective view of the backside of the backer component showing the handle component partially folded outwardly about the long axis thereof for gripping purposes.

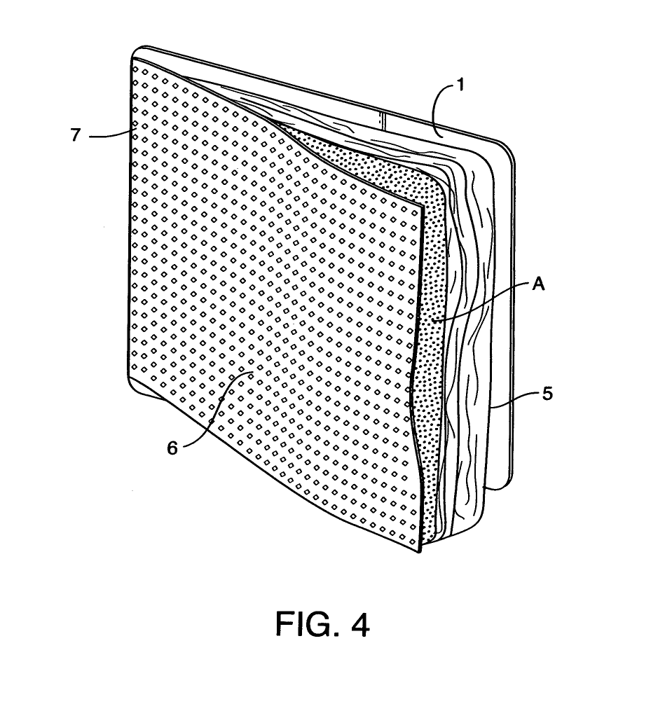

4. FIG. 4 is a perspective view of the preferred embodiment of the application device showing the front side of the backer component, first piece component and frontal membrane component adhesively all affixed to one another but just on one side and with powder material therebetween being also shown.

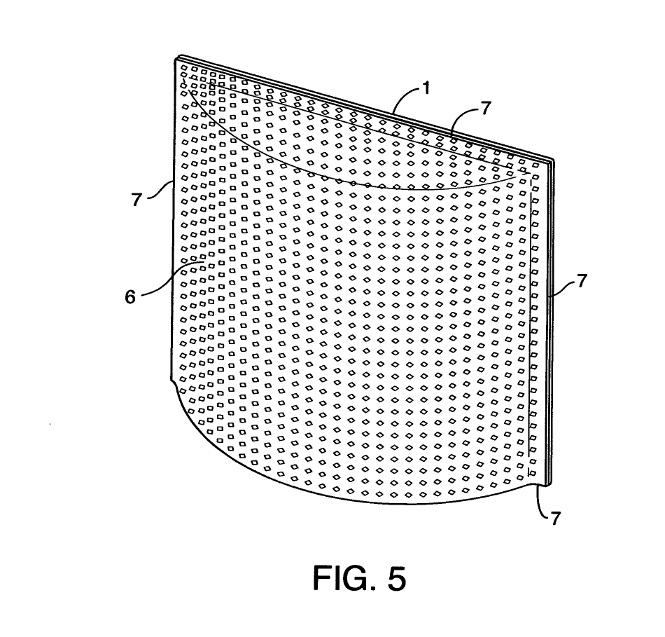

5. FIG. 5 is an isolated perspective view of the first embodiment of the application device in an intact fully assembled state.

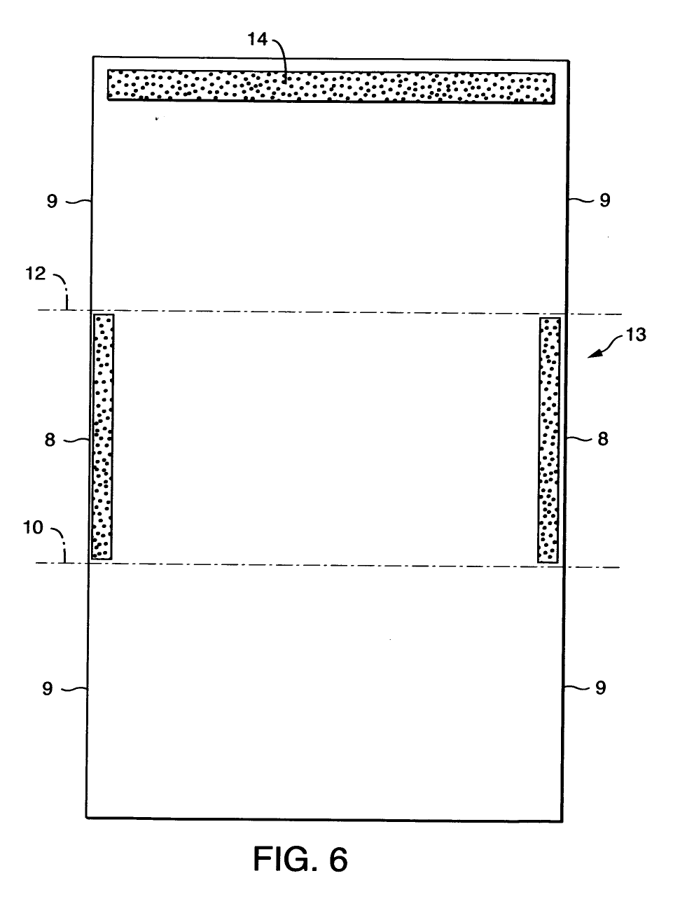

6. FIG. 6 shows, in frontal plan view, a paper pocket unit prior to assembly thereof.

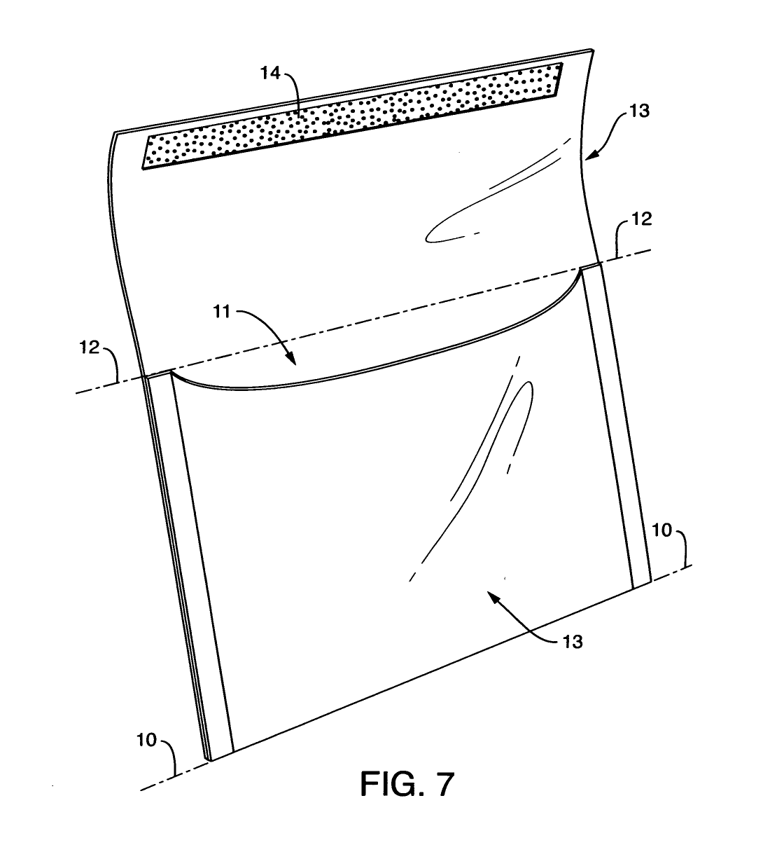

7. FIG. 7 shows in perspective view, the fully assembled paper pocket unit.

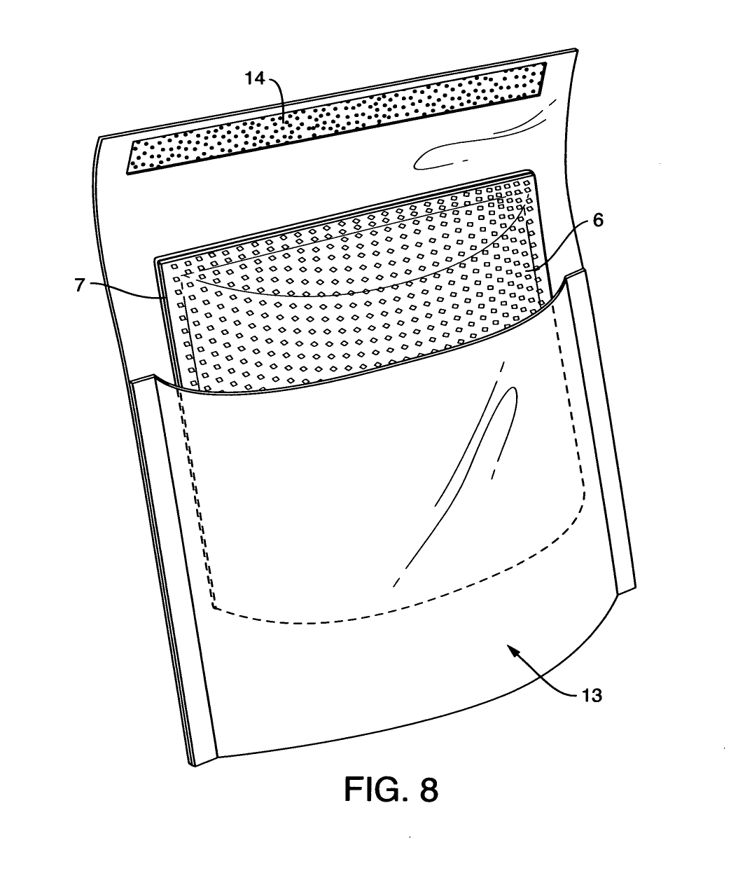

8. FIG. 8 shows, in frontally oriented perspective view, the invention being an intact, fully assembled application device being partially held within a paper pocket unit.

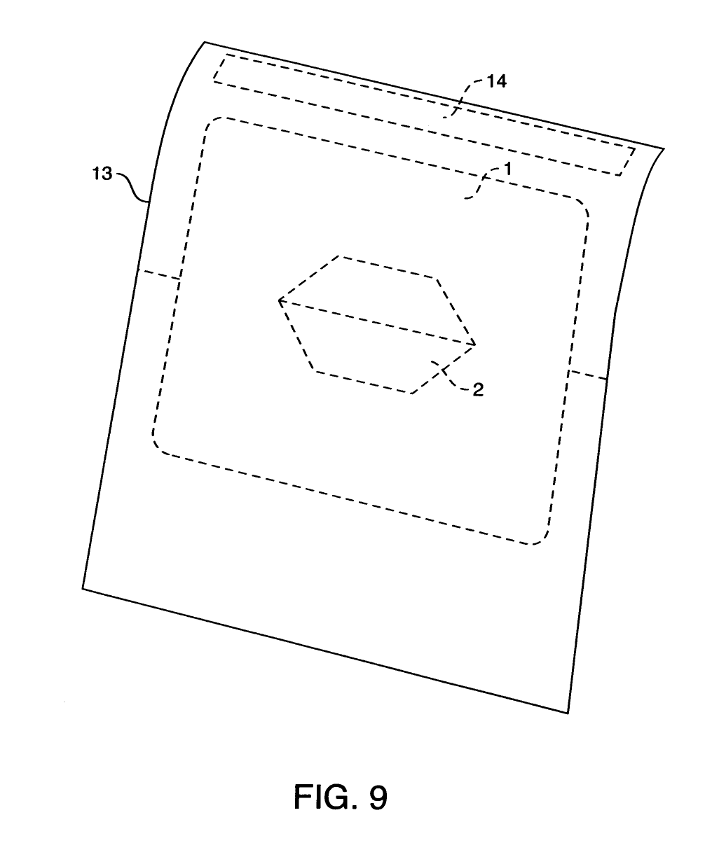

9. FIG. 9 shows, in posteriorly oriented perspective view, the invention being an intact, fully assembled application device being partially held within the paper pocket unit.

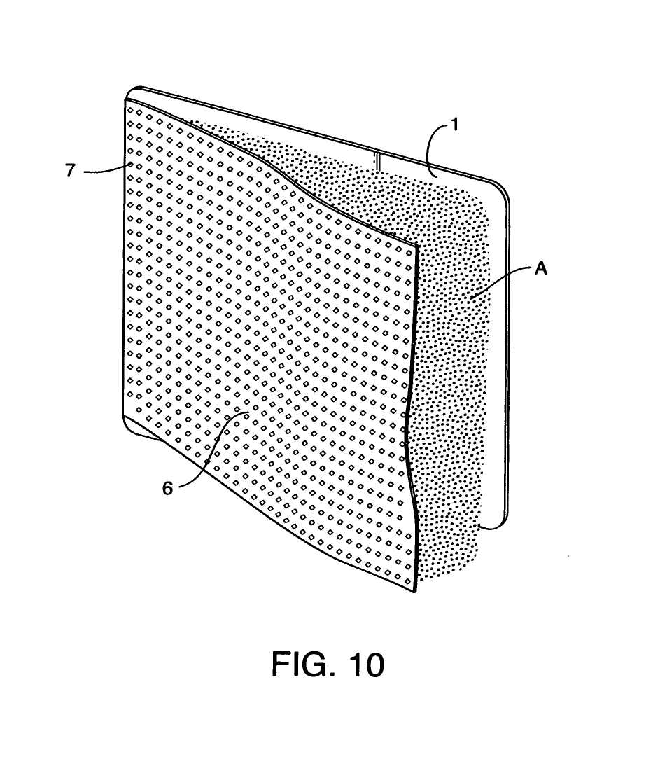

10. FIG. 10 is a perspective view similar in nature to the view as shown in FIG. 4, but rather of the second embodiment of the application device.

### A DESCRIPTION OF THE PREFERRED EMBODIMENTS

FIG. 1 shows the intact invented powder application device being held by thumb and forefinger of the hand B of a person seeking to utilize the invention. The invention is grasped as shown in FIG. 1 by way of so taking hold of the handle component 2 thereof such as is affixed as seen in FIG. 2 to the cardboard backer component 1 of the invention. Handle component 2 is partially affixed to the posterior side of component 1 by way of first affixation means 3 which could be glue sealant material as seen in FIG. 3 or a piece of tape with adhesive substance on both sides, what can be termed as being double-sided adhesive tape. FIG. 3 shows the non-affixed portion of handle component 2 folded outwardly about its long axis 4; the outwardly folded portion thereof being what is gripped or held as shown in FIG. 1. FIG. 4 shows a first piece component 5 of the invention in apposition to the anterior walling of backer component 1 as well as frontal membrane component 6 positioned anteriorly to first piece component 5 with powder material A sandwiched in between components 5 and 6. Component 5 is made up of a garden variety cotton batten material. Component 6 is made up a permeable, membranous material sufficiently porous to enable passage through frontal membrane component 6 of grains of powder material A but only when the fully intact invention is pressed against a portion of the skin surface of a person's body. Powder material A is a material with grains thereof being the order of size of grains of a cornstarch based powder material. Frontal membrane component 6 is typically made up of the same membranous material such as forms the outer layer of a SURGIPAD® surgical dressing unit such as is manufactured and sold by Johnson and Johnson. FIG. 4 shows all of the component parts of the preferred embodiment of the powder application device except as respects the handle component 2 thereof, all in a partially dissembled state for illustrative purposes and in apposition to one another. FIG. 4 also evidences the manner in which all of the component parts of the application device are affixed to one another by way of resort to second affixation means 7 as shown on one side only of FIG. 4 and which could be a glue sealant material or a double-sided adhesive tape material such as was earlier described. An alternate embodiment of the application device portion of the invention as distinguished from the above-described preferred embodiment thereof, is one as shown in a partially dissembled state in FIG. 10 wherein there is no first piece component 5 but rather only a frontal membrane component 6 affixed, in the manner as noted above, anteriorly to backer component 1 with powder material A sandwiched in between the anterior side of backer component 1 and the posterior side of frontal membrane component 6. FIG. 5 is an isolated perspective view of the first embodiment of the intact fully assembled powder application device featuring second affixation means 7 about all sides thereof. FIG. 6 shows in plan view, an unassembled optional paper pocket unit 13 meant ultimately to hold the powder application device, and FIG. 7 shows paper pocket unit 13 fully assembled. FIGS. 8 and 9 each show in perspective views, anteriorly oriented and then posteriorly oriented, the manner in which the intact biodegradable powder application device is amenable to being held within an assembled pocket unit 13. FIG. 6 shows in plan view, pocket unit 13 with applied lateralized third affixation means 8, e.g., glue sealant material or strips of double sided tape material prior to assembly thereof. Pocket unit 13 is assembled simply by first applying third affixation means 8 to the lateral edges of the middle one-third or so portion of the piece of paper material 9 selected for purposes of assembling pocket unit 13. The lower one-third or so portion of paper material 9 is then simply folded upwardly about a first horizontally inclined transverse axis 10 thereof and located coincident with bottom edging of third affixation means 8 and the so folded lower portion is then affixed to the middle portion via third affixation means 8. Such folding and affixation results in the formation of a pocket 11 seen in FIG. 7 within pocket unit 13 into which the intact powder application device can then be placed, with that upper portion of paper material 9 located superiorly to a second horizontally inclined transverse axis 12 located coincident with the uppermost edgings of third affixation means 8 being foldable and unfoldable about the application device located within the pocket 11 portion of the thence assembled pocket unit 13. Fourth affixation means 14 also seen in FIG. 7, being glue sealant material or such double-sided adhesive tape positioned near the uppermost frontal edging of paper material 9 can serve to seal the application device within the pocket 11 of pocket unit 13 simply by folding the upper portion of paper material 9 downwardly about axis 12 resulting in a sealing of the intact powder application device within pocket 11 by way of pressing the top edging of paper material 9 and affixation means 14 as against the lower portion of the outer walling of created pocket unit 13. As previously noted, FIGS. 8 and 9 show the powder application device being partially held within pocket 11 of pocket unit 13.

Also as previously noted, the powder application device and the pocket unit 13 are wholly biodegradable. The materials required for complete assembly of the application device and pocket unit 13 are relatively inexpensive. The device provides a quick and ready way for applying powder to a distressed portion of the skin surface of a person's body so as to both avoid clumping or wasteful dispersion of the powder once so applied. Considerations involving relative ease of and efficacy in respect of powder application are readily appreciated as regards the appropriate treatment of rashes and bedsores afflicting many bedridden hospital and nursing home patients. The application device once removed from pocket unit 13 and then used in the application of powder A to the needed portion of a patient's skin, is simply reinserted into pocket unit 13 after such use and then discarded after pocket unit 13 is resealed by again pressing affixation means 14 as against the bottom most frontal portion of pocket unit 13.

In closing, respectfully submitted, the instant invention as described above is not only new, useful and unique in respect of the art involving devices serving to facilitate the application of powders to an afflicted portion of the skin surface of a person's body, but rather, it is also, indeed, veritably revolutionary within the field of such art.

What is claimed is:

1. A pocketable biodegradable powder application device, comprising:
a. a cardboard backer component;
b. a cardboard handle component partially affixed with first affixation means to a posterior side of said backer component;
c. a non-affixed portion of said handle component, foldable about a long axis thereof and being amenable to being gripped by one of a person's thumbs and adjacent forefinger;
d. a first piece component made of cotton batten material;
e. said first piece component being positioned adjacent an anterior side of said backer component;
f. a frontal membrane component;
g. said frontal membrane component being positioned adjacent an anterior side of said first piece component;
h. said backer component, said first piece component and said frontal membrane component being all affixed to one another about all outer perimeters of each with second affixation means in assembly of an intact powder application device, and;
i. said frontal membrane component being made of a permeable, membranous material sufficiently porous to enable passage through said frontal membrane component of grains of cornstarch based powder material but only when said powder application device is pressed against a portion of the skin surface of a person's body.

2. The pocketable biodegradable powder application device of claim 1, whereby said first affixation means is a glue sealant material.

3. The pocketable biodegradable powder application device of claim 1, whereby said first affixation means is double sided adhesive tape material.

4. The pocketable biodegradable powder application device of claim 1, whereby said second affixation means is a glue sealant material.

5. The pocketable biodegradable powder application device of claim 1, whereby said second affixation means is double sided adhesive tape material.

6. The pocketable biodegradable powder application device of claim 1, whereby said frontal membrane component is made up of the same membranous material such as forms the outer layer of a SURGIPAD® surgical dressing unit.

7. A pocketable biodegradable powder application device, comprising:
a. a cardboard backer component;
b. a cardboard handle component partially affixed with first affixation means to a posterior side of said backer component;
c. a non-affixed portion of said handle component, foldable about a long axis thereof and being amenable to being gripped by one of person's thumbs and adjacent forefinger;
d. a frontal membrane component;
e. said frontal membrane component being positioned adjacent an anterior side of said backer component;
f. said backer component and said frontal membrane component being affixed to one another about all outer perimeters of each with second affixation means in assembly of an intact powder application device, and;
g. said frontal membrane component being made of a permeable, membranous material sufficiently porous to enable passage through said frontal membrane component of grains of cornstarch based powder material but only when said powder application device is pressed against a portion of the skin surface of a person's body.

8. The pocketable biodegradable powder application device of claim 7, whereby said first affixation means is a glue sealant material.

9. The pocketable biodegradable powder application device of claim 7, whereby said first affixation means is double sided adhesive tape material.

10. The pocketable biodegradable powder application device of claim 7, whereby said second affixation means is a glue sealant material.

11. The pocketable biodegradable powder application device of claim 7, whereby said second affixation means is double sided adhesive tape material.

12. The pocketable biodegradable powder application device of claim 7, whereby said frontal membrane component is made up of the same membranous material such as forms the outer layer of a SURGIPAD® surgical dressing unit.

13. A pocketable biodegradable powder application device, comprising:
a. a cardboard backer component;
b. a cardboard handle component partially affixed with first affixation means to a posterior side of said backer component;
c. a non-affixed portion of said handle component, foldable about a long axis thereof and being amenable to being gripped by one of a person's thumbs and adjacent forefinger;
d. a first piece component made of cotton batten material;
e. said first piece component being positioned adjacent an anterior side of said backer component;
f. a frontal membrane component;
g. said frontal membrane component being positioned adjacent an anterior side of said first piece component;
h. said backer component, said first piece component and said frontal membrane component being all affixed to one another about all outer perimeters of each with second affixation means in assembly of an intact powder application device;
i. said frontal membrane component being made of a permeable, membranous material sufficiently porous to enable passage through said frontal membrane component of grains of cornstarch based powder material but only when said powder application device is pressed against a portion of the skin surface of a person's body;
j. a piece of rectangularly shaped paper material;
k. a lower nearly one-third portion of said piece of paper material being upwardly folded about a first horizontally inclined transverse axis thereof;
l. third affixation means being applied to lateral edges of a middle nearly one-third portion of said piece of paper material with bottom ends of said lateral edges being coincident with said first axis;
m. said third affixation means serving to hold lateral edging of said lower portion to said lateral edges of said middle portion to thereby create a formed pocket within an open fully assembled pocket unit, amenable to receipt of a fully intact said powder application device;
n. an upper portion of said piece of paper material being downwardly folded about a second horizontally inclined transverse axis thereof, with upper ends of said lateral edges being coincident with said second axis;
o. fourth affixation means being applied near frontal uppermost edging of said piece of paper material, and;
p. said fourth affixation means serving to hold said upper portion, when so downwardly folded, fast against outer walling of said formed pocket in respect of enclosing within a closed said fully assembled pocket unit, said powder application device.

14. The pocketable biodegradable powder application device of claim 13, whereby said first affixation means is a glue sealant material.

15. The pocketable biodegradable powder application device of claim 13, whereby said first affixation means is double sided adhesive tape material.

16. The pocketable biodegradable powder application device of claim 13, whereby said second affixation means is a glue sealant material.

17. The pocketable biodegradable powder application device of claim 13, whereby said second affixation means is double sided adhesive tape material.

18. The pocketable biodegradable powder application device of claim 13, whereby said third affixation means is a glue sealant material.

19. The pocketable biodegradable powder application device of claim 13, whereby said third affixation means is double sided adhesive tape material.

20. The pocketable biodegradable powder application device of claim 13, whereby said fourth affixation means is a glue sealant material.

21. The pocketable biodegradable powder application device of claim 13, whereby said fourth affixation means is a double sided adhesive tape material.

22. The pocketable biodegradable powder application device of claim 13, whereby said frontal membrane component is made up of the same membranous material such as forms the outer layer of a SURGIPAD® surgical dressing unit.
